(19) United States
(12) Patent Application Publication
(10) Pub. No.: US 2010/0215713 A1
(43) Pub. Date: Aug. 26, 2010

(54) SCAFFOLDS FOR FOLLICLE TRANSPLANTATION

(75) Inventors: Marie-Madeleine Dolmans-Van Der Vorst, Kraainem (BE); Christiani Andrade Amorim, Bruxelles (BE); Anne Van Langendonckt, Bruxelles (BE); Jacques Donnez, Bruxelles (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Catholique de Louvain

(21) Appl. No.: 12/681,075

(22) PCT Filed: Sep. 30, 2008
(86) PCT No.: PCT/EP08/63067

§ 371 Date: Mar. 31, 2010

(30) Foreign Application Priority Data
Oct. 1, 2007 (EP) 07117661.4

(51) Int. Cl. A61F 2/00; A61K 38/22; A61K 38/28; A61K 38/43; A61K 31/34; A61K 31/355; A61K 31/522; A61K 38/20; A61P 15/08

(52) U.S. Cl. 424/423; 514/12; 514/3; 424/94.1; 514/474; 514/458; 514/263.34; 424/85.2

(57) ABSTRACT

The present invention provides for a device comprising a scaffold composition, a bioactive composition and a bio-in-hibiting composition, wherein said bioactive and bio-inhibiting compositions are incorporated into or coated onto said scaffold composition, wherein said scaffold composition temporally supports survival and growth of resident follicles, migration and multiplication of stroma cells and spreading and organization of endothelial cells and new vessels wherein said bioactive composition regulates development of a resident follicle, formation of new blood vessels and chemoattraction and proliferation of stroma cells and wherein the bio-inhibiting composition regulates inhibition of the development of a second resident follicle. The presence of the bio-inhibiting composition within the scaffold is involved in the quiescence of the follicles in the primordial stage, which is important to restore fertility.

### FIELD OF THE INVENTION

The present invention relates to devices or vehicles to graft isolated ovarian follicles or small fragments of ovarian tissue back to the patient after radio-or chemotherapeutic anti-cancer treatment, capable of restoring normal ovarian function with hormone production and fertility.

### BACKGROUND OF THE INVENTION

Recent progress in oncology has significantly increased the long-term survival rate of cancer patients. Unfortunately, for women, cancer treatments such as chemo/radiotherapy can be very harmful to the ovaries, frequently resulting in loss of both endocrine and reproductive functions. For these patients, who originally had expectations of a normal reproductive lifespan, the realization that they might suffer a premature menopause, with its symptoms, signs and devastating consequence, can have a profound impact on their self-esteem and quality of life. Hence, in the last years, alternatives have been studied to re-establish normal ovarian function and fertility in cancer patients. Prior to the initiation of cancer treatment, it is possible to retrieve and cryopreserve ovarian tissue containing the primordial follicles and after the disease remission, they can be transplanted back enclosed in the ovarian tissue or isolated.

Reintegration of cryopreserved ovarian tissue has however two serious drawbacks, one of them being that one first has to ascertain that absolutely no malignant cells are present or remaining in the ovarian tissue before reintegration into the patient. When the risk of reintegrating malignant cells is too high, the technique can simply not be used safely. A second problem is that reintegration of ovarian tissue often leads to a high loss of individual primordial follicles due to ischaemia before the revascularisation or neovascularisation process in the patient's tissue is completed. In addition, since the distribution of primordial follicles in human ovaries seems to be irregular, it is not possible to guarantee the presence or to know the number of follicles in the ovarian graft that are able to maturate after reintegration in the patient. The amount of viable primordial follicles that can develop into a mature follicle is often very small, resulting in a low chance of actually getting pregnant after transplantation.

To avoid such drawbacks, these primordial follicles could be in vitro cultured. They would have also their oocyte matured and fertilized in vitro, and the resulted embryo could be transferred to the mother. However, in vitro development of human primordial follicles has certainly proved challenging. Since the time required for follicles to grow is so long in humans (up to 120 days) and the precise mechanism involved in this process is unknown, this possibly discourages researchers from conducting studies in this area and so far this alternative did not offer any successful results.

Another alternative could be grafting of isolated follicles. This procedure has been proven successful, since isolated primordial follicles transplanted in plasma clot were able to develop until antral stage. In addition, this grafting protocol also allowed the formation of a stromal-like structure, with cell organization and vascularisation similar to a normal ovary. However, the drawback of this technique is the difficulty to recover the plasma clot with the follicles and the high concentration of serum, which is toxic to the primordial follicle cells.

The main aim of the present invention is to provide for a device or a vehicle to graft isolated ovarian follicles or small fragments of ovarian tissue back to the patient after cancer remission, overcoming the above stated problems with the known techniques. Furthermore, the scaffold should not act only as a vehicle, but also as a temporary surrogate for native extracellular matrix, allowing the survival and growth of human ovarian follicles. It can also help to induce the formation of an ovarian-like structure, favouring cell migration, attachment, multiplication and vascularisation. In addition, this scaffold must permit transport of oxygen, nutrients and degradation products, it should permit grafting in different sites of the patient, be biocompatible and biodegradable and easily fabricated into a variety of sizes and shapes with several pore sizes and interconnectivity in order to choose the best correlation between material degradation, follicle development, cell migration and proliferation and patient response. In addition, they should have adequate mechanical properties to match the intended site of implantation and handling and be able to carry a higher number of follicles.

### SUMMARY OF THE INVENTION

The present invention provides for a device comprising a scaffold composition, a bioactive composition and a bio-inhibiting composition, wherein said bioactive and bio-inhibiting compositions are incorporated into or coated onto said scaffold composition, wherein said scaffold composition temporally supports survival and growth of resident follicles, migration and multiplication of stroma cells and spreading and organization of endothelial cells and new vessels wherein said bioactive composition regulates development of a resident follicle, formation of new blood vessels and chemoattraction and proliferation of stroma cells and wherein the bio-inhibiting composition regulates inhibition of the development of a second resident follicle. The presence of the bio-inhibiting composition within the scaffold is involved in the quiescence of the follicles in the primordial stage, which is important to restore fertility.

The ovarian follicle is a very particular structure that can increase its size about 600 times during folliculogenesis (primordial follicle: 30 μm—Graafian follicle: 18000 μm). It comprises two types of cells: granulosa cells and oocytes, which have different origins and requirements. Follicular growth requires a plethora of autocrine, paracrine and endocrine factors during different stages of development (most of these factors as well as their mechanisms of action remain unknown). Therefore, vascularisation and stroma cells play an essential role in folliculogenesis. Consequently, it is very important to have all these features in mind during the design and experimentation of the scaffold (vehicle).

The device could for example be of a cylindrical shape comprising an inner tube comprising pores (size of an immature follicle approx. 30 μm) for the introduction of isolated ovarian follicles or small parts of ovarian tissue, which is closed after the introduction of said follicles thereby restraining the follicles in the cylindrical device until maturation is completed. The device comprises between the outer cylinder and the inner tube a meshwork of scaffolds acting as a temporary surrogate for the native extracellular matrix and helping the formation of an ovarian-like structure, favouring cell migration, attachment, multiplication and vascularisation. In addition, the scaffold permits transport of oxygen, nutrients and degradation products. Prior to the implantation of the device into the remaining female ovary, the device could be cultured in vitro performing a rolling movement allowing the isolated follicles to enter the meshwork of scaffolds, attach to it and develop or at least remain viable, using appropriate culturing conditions. The cylindrical device should preferentially also comprise a gradient of the bio-activating and bio-inhibiting factors listed further down in the application in order to create a kind of time-gradient of follicle development. The goal of this gradient is to induce the maturation of only one or very few primordial follicle(s) in the device at the time and preventing the maturation of the remaining follicles in the device in order to really restore long-term fertility of the patient after the device is reincorporated in the remaining ovarian organ of the patient. part of the bio-activating factors also promote the formation of new blood vessels, required for further transport of oxygen, nutrients and degradation products, and allow the migration and proliferation of stroma cells from the remaining ovarian tissue of the patient to the scaffold in order to create a new ovarian-like structure.

The invention thus provides a solution to the problem posed in the prior art techniques. The big difference of the scaffold system of the invention with those of the prior art is that the follicles are able to receive all needed factors for development. This is for example done by inducing neo-vascularisation inside the scaffold, enabling the transport of the plethora of (many yet unknown) factors and stimulants needed for efficient follicle development and maturation. The scaffold system of the invention is biodegradable and biocompatible and can be implanted in the patient. After neo-vascularisation, all naturally present and yet largely unknown factors and signals are transported right to the follicles inside the scaffold, which cannot be mimicked in any in vitro model system provided in the prior art.

The invention therefore provides a device, comprising a scaffold composition consisting essentially of a flexible implantable biocompatible matrix with a porous structure, a bio-activating composition and a bio-inhibiting composition, wherein said bio-activating and bio-inhibiting composition are incorporated into or coated onto said scaffold composition, wherein said scaffold composition is biocompatible and biodegradable and temporally controls growth of resident primordial follicles, migration and multiplication of stroma cells and spreading and organization of endothelial cells and new vessels, wherein said bio-activating composition regulates positive development of said resident primordial follicles into primary follicles, formation of new blood vessels and chemoattraction and proliferation of stroma cells and wherein the bio-inhibiting composition inhibits the development of other resident primordial follicles into primary follicles. Preferably, said bio-activating composition and said bio-inhibiting composition are extracellular matrix components. In a further preferred embodiment, the bio-activating composition and/or the bio-inhibiting composition are encapsulated within a slow release container. In a further preferred embodiment, the bio-inhibiting composition comprises anti-Müllerian hormone (AMH) and/or stromal cell-derived factor 1 (SDF-1). In a further preferred embodiment, the bio-activating composition comprises growth differentiation factor-9 (GDF-9).

In an alternative embodiment of the device according to the invention, the bio-activating composition comprises one ore more of activin, basic fibroblast growth factor (bFGF), Kit ligand, insulin, bone morphogenetic protein-4 (BMP-4), bone morphogenetic protein—7 (BMP-7), leukaemia inhibitory factor (LIF), nerve growth factor (NGF) and keratinocyte growth factor (KGF), 17α hydroxylase (17α-OH). In addition, the device of the invention can further comprise one ore more of factors reducing ischaemic damages such as ascorbic acid, vitamin E or Pentoxifylline.

In an alternative embodiment of the device according to the invention, the bio-activating composition comprises one ore more of factors involved in angiogenesis such as vascular endothelial growth factor (VEGF), platelet-derived growth factor, angiopoietins such as Angiopoietin-1, placenta growth factor (PIGF), HIF polyl hydroxylases (PHD1) and hypoxia mimic ions, PR39, p53, interleukin-8 (IL-8), transforming growth factor-β1 (TGF-β1) and nitric oxide (NO).

In a preferred embodiment of the device according to the invention, at least one member of each of the following groups of factors is present:

a) factors involved in the primordial follicle or preantral development such as: activin, Basic fibroblast growth factor (bFGF), Kit ligand, Insulin, Bone morphogenetic protein—4 (BMP-4), Bone morphogenetic protein—7 (BMP-7), Leukaemia inhibitory factor (LIF), Nerve growth factor (NGF), Keratinocyte growth factor (KGF), Growth Differentiation Factor-9 (GDF-9) or 17α hydroxylase (17α-OH);

b) negative regulators of early follicle development: Anti-Müllerian Hormone (AMH) and/or stromal cell-derived factor 1 (SDF-1);

c) optionally, factors that reduce ischaemic damages such as Ascorbic acid, Vitamin E, or Pentoxifylline;

d) factors involved in angiogenesis such as: Vascular endothelial growth factor (VEGF), Platelet-derived growth factor, Angiopoietins, Angiopoietin-1, Placenta growth factor (PIGF), HIF polyl hydroxylases (PHD1), Hypoxia mimic ions, PR39, p53, Interleukin-8 (IL-8), Transforming Growth Factor-β1 (TGF-β1) and Nitric Oxide (NO).

More preferably, the following factors are present in the device of the invention in combination: one or more factors involved in the primordial follicle development selected from GDF-9 and/or 17α-OH; one or more negative regulators of early follicle development selected from Anti-Müllerian Hormone (AMH) and/or stromal cell-derived factor 1 (SDF-1); one or more factors that reduce ischaemic damages; and one or more factors involved in angiogenesis.

In the most preferred embodiment of the device of the invention, the following factors are present in combination: Growth differentiation factor—9 (GDF-9), Anti-Müllerian Hormone (AMH), Ascorbic acid and HIF polyl hydroxylases (PHD1).

In certain embodiments, the device of the invention comprises a scaffold composition comprising pores having a pore size between 10 and 6000 pm and/or wherein the pores are distributed within the scaffold in a controlled pattern, whereby the pores in the region of the centre of the scaffold are wider than the pores in the region towards the outer surface of the scaffold.

In further embodiments, the device of the invention is provided with an inlet for the introduction of the follicles in the scaffold and/or, whereby the flexible implantable biocompatible matrix has a sufficient elasticity to allow follicle growth within the scaffold allowing the pores to adjust during growth from 10 to 6000 μm and/or wherein said device is cylindrical or suitable for use in a rolling-culture process in vitro.

In further embodiments, the device of the invention further comprises follicles.

In further embodiments, the device of the invention is constructed out of biodegradable material selected from the group consisting of: linear aliphatic polyesters: poly(lactic acid)—PLA, poly(glycolic acid)—PGA, poly(caprolactone)—PCL, poly(hydroxy butyrate)—PHB, including homopolymers and copolymers thereof, polyanhydrides, Poly(propylene fumarates) (PPF), Tyrosine-derived polymers, poly(ortho esters), poly(anhydrides), polyphosphazenes, polyurethanes, hydrogel matrices, alginic acid, hyaluronic acid, poly(γ-glutamic acid), amphiphiles, or combinations thereof.

### DETAILED DESCRIPTION OF THE INVENTION

To develop the device and the scaffold according to the present invention, the device has to have an adequate (1) scaffold degradability in vivo; (2) scaffold compatibility with the patient as well as with the ovarian follicle growth, (3) scaffold bioactivity to regulate development of ovarian follicles (e.g. the provision of nutrients, growth factors, oxygen, formation of blood vessels and migration and proliferation of stroma cells) and (4) short-term and long-term survival of the ovarian follicles comprised in the grafted scaffold.

The scaffold degradability can be established by choosing the appropriate (bio)polymers.

Different polymers such as linear aliphatic polyesters: poly(lactic acid)—PLA, poly(glycolic acid)—PGA, poly(caprolactone)—PCL, poly(hydroxy butyrate)—PHB, including homopolymers and copolymers thereof, can be used. These biodegradable, thermoplastic polyesters are characterized by degradation times ranging from days to years depending on the formulation and initial molecular weight. PLA, PGA and PCL are derived from three monomers: lactide, glycolide, and caprolactone. One of the main advantages of PLA, PGA and their copolymers is that their degradation products are natural metabolites (lactic acid and glycolic acid) which are removed from the body by normal pathways. Lactic acid enters tricarboxylic acid cycle and is excreted as water and carbon dioxide and glycolic acid also can be excreted by urine. PCL degrades at a significantly slower rate, but PCL-based copolymers have recently been synthesized to improve degradation properties. PHB and its copolymers degrade very slowly due to their hydrophobic nature.

Other suitable synthetic degradable polymers could be polyanhydrides, a class of biodegradable polymers characterized by the hydrolic instability of anhydride bonds that degrades rapidly to form non-toxic monomers. This degradation can be controlled by manipulation of the polymer composition.

Poly(propylene fumarates) (PPF) could also be used. They can degrade through hydrolysis of the ester bonds similar to glycolide and lactide polymers.

Tyrosine-derived polymers could also be used since it has been shown that they have promising biocompatibility and represent one of the new second generation biomaterials.

Alternatively, poly(ortho esters)—like poly(anhydrides) could be used. These were developed to address the issue of surface erosion to improve the release of drugs from erodible matrices and have therefore been extensively developed for applications in drug delivery and they will probably play an important role in tissue engineering scaffolding.

Also, polyphosphazenes could be used. They consist of several different polymers with general common structure that can be biodegradable with incorporation of specific side groups. Similarly to polyanhydrides and poly(ortho esters), they have been frequently used for controlled drug delivery applications and they also have been explored for tissue engineering scaffolding applications.

Also, polyurethanes could be used for the scaffold allowing the structural variations to achieve a range of mechanical properties. Due to their structure/property diversity, they are considered as one of the most bio- and blood-compatible materials known today.

Finally, as an alternative to the use of chemical polymers, hydrogel matrices known to have excellent 3D culture properties could be useful as a scaffold because of its ability to mimic the 3D structure of the ovary, needed for follicle cells to remain viable and to develop. Different acids, such as alginic acid, hyaluronic acid and poly(γ-glutamic acid), and some molecules, such as peptides amphiphiles, can be used to form the hydrogels.

In order to improve follicle adhesion to the scaffold, its surface can be modified with adhesion promoting molecules and/or substances, such as: laminin, fibronectin, collagen, gelatin, chitosan or fibrinogen.

The scaffold manufacturing is another important issue that must be taken into consideration. The fabrication approaches must not only replicate the properties of the organ (ovary) at the macroscopical level, but also recreate the nanoscale details observed in the real tissue at the cellular level. The dimensions of the extracellular matrix fibres and basement membranes, and their interconnecting nanopores found in the natural tissue typically have nanoscaled dimensions. A list of different techniques that can be used is provided hereunder:
Gas foaming—a biodegradable polymer is saturated with carbon dioxide (CO₂) at high pressures. The solubility of the gas in the polymer is then decreased rapidly by bringing the CO₂ pressure back to atmospheric level. This results in nucleation and growth of gas bubbles.
Fibre bonding/fibre meshes—it increases the mechanical properties of the scaffolds by dissolving the PLA and casting over PGA mesh. The solvent is allowed to evaporate and the construct is then heated above the melting point of PGA. Once the PLA-PGA construct has cooled, the PLA is removed by dissolving it again. This treatment results in a mesh of PGA fibres joined at the cross-point.
Phase separation—the polymer solution separates into two phases, a polymer-rich phase and a polymer-lean phase. After the solvent is removed, the polymer-rich phase solidifies. Biologically active molecules can be added to the polymer solution.
Melt moulding—one of the techniques involved in this process involves filling a Teflon mould with polymer powder and gelatine microspheres, of specific diameter, and then heating the mould above the glass-transition temperature of the polymer while applying pressure to the mixture. This treatment causes the polymer particles to bond together. Once the mould is removed, the gelatine component is leached out by immersing in water and the scaffold is then dried.
Emulsion freeze-drying—this process involves adding ultrapure water to a solution of methylene chloride with PGA. The two immiscible layers are then homogenised to form a water-in-oil emulsion, which is then quenched in liquid nitrogen and freeze-dried to produce the porous structure.
Freeze drying—the polymer is dissolved in glacial acetic acid or benzene and the resultant solution is frozen and freeze-dried to yield porous matrices.
Solution casting—PGLA is dissolved in chloroform and then precipitated by the addition of methanol before the material is pressed into a mould and heated to 45-48° C. for 24 hours.
Solid freeform fabrication techniques (also known as rapid prototype)—it is a group of computer-controlled fabrication techniques that allows complex scaffold designs to be realized, with localized pore morphologies and porosities and incorporated bioactive molecules to suit the requirements of the cells. The general process involves producing a computer-generated model using computer-aided design (CAD) software. This CAD model is then expressed as a series of cross-sectional layers. The data is implemented to the solid freeform fabrication machine, which produces the physical model.
Indirect solid freeform fabrication technique—in this procedure, a negative mould is generated by one of the solid freeform fabrication techniques and then the scaffold is formed by adding the casting solution to the negative mould using the desired polymer. After solidification, the negative mould is removed by dissolution, melting to other procedures.
Particulate-leaching—in this technique, salt is first ground into small particles and those of the desired size are transferred into a mould. A polymer solution is then cast into the salt-filled mould. After the evaporation of the solvent, the salt crystals are leached away using water to form the pores of the scaffold.
Electrospinning—it is a process capable of producing ultra-fine fibres by electrically charging a suspended droplet of polymer melt or solution.
Vibrating particle fabrication technique—in this process, the polymer is dissolved in solvent and the solution is mould with salt particles. The particles are dispersed using vortex and at predetermined time intervals, more particles are added. Then, the solution evaporates under continuous vibration and the scaffold is subjected to heat and vacuum.

Use of each one of the above polymers or techniques, or combinations of several of these polymers and/or techniques offers the possibility to mould the scaffold varying some of the most important parameters: porosity, pore size distribution, orientation and interconnectivity, which can positively affect cell distribution and mass transport of soluble signalling molecules, nutrients, metabolic waste removal, tissue integration and neovascularisation and follicular development.

The scaffolds can be cast in different shapes and sizes and with several pore sizes and interconnectivity in order to choose the best correlation between material degradation, follicle development, cell migration and proliferation and patient response. Other parameters such as adequate mechanical properties to match the intended site of implantation and handling and ability to carry out a higher number of follicles can also be taken into consideration. In order to test the degradability and biocompatibility of the scaffold several experiments have to be carried out.

Testing of in Vitro Degradation Kinetics

Scaffold incubation—Scaffolds fabricated with one of the above mentioned polymers and techniques are immersed in 30 ml PBS (pH 7.4) and stirred in a thermostat at 15 rpm and 37° C. Degradation behaviour is assessed after different time periods: 0 (control), 1, 2, 3, 4, and 6 weeks. After every one of these periods, samples are removed, air-dried overnight and vacuum-dried for 24 hours in order to perform the following analysis:

Molecular weight—Changes in the weight average molecular weight of the polymer is determined as a function of degradation time using gel permeation chromatography (GPC) equipped with a refractive index detector. The dried samples are dissolved in tetrahydrofuran at a concentration of 8 mg/ml and eluted through the column at a flow rate of 1 ml/min at 37° C. Polystyrene standards are used to obtain a primary calibration curve. All samples of the same polymer type are analysed at a single run.

Weight and thickness—Before drying the samples, the wet weight and thickness are measured in order to determine the medium absorption of the scaffolds, which is calculated using the following formula: Medium absorption=

$$\frac{( {W}_{f , w} - {W}_{f} )}{{W}_{f}} ,$$

where: W_{f,w}—wet weight; W_{f}—final dry weight.

The normalized weight and thickness of the degraded dried scaffolds are calculated by W_{f}/Wᵢ [Wₒ—initial weight (week 0)] and d_{f}/dᵢ [dₒ—initial thickness (week 0)], respectively.

Morphology analysis—Micrographs are obtained in a scanning electron microscope (SEM) to study temporal, microscopic, structural changes in the scaffolds as they degrade over time. For that, the dried samples are gold coated using a sputter coater set at 20 mA for a total time of 120 seconds (coating thickness, approximately 40 nm). Then, they are imaged with a scanning electron microscope operated at 20 kV.

pH test—In order to determine the effect of degradation on the pH around the scaffolds, the scaffolds are divided in two groups: in the first group, PBS is changed every 24 hours and in the second, the buffer solution is not changed. Samples of PBS are taken at the beginning of every week in order to assess PBS pH.

Testing of in Vivo Degradation Kinetics and Scaffold Toxicity Assay

Scaffold implantation in sheep—The sheep has been chosen as experimental model mainly owing to the similarities of their ovaries to those of humans: sheep ovaries have almost the same size and stroma composition and similar follicle size and growth patterns. Scaffolds fabricated with one of the above-mentioned polymers and techniques are implanted in the sheep ovary, according to Donnez et al. (Lancet, 364:1405-1410, 2004). Briefly, a laparotomy is performed and two windows are created beneath the ovarian hilus, close to the ovarian blood vessels. Alternatively, the scaffolds can also be placed in the intraovarian area as described by Donnez et al. (Hum Reprod, 21:183-188, 2006). One scaffold is first sandwiched between two nitrocellulose filters to block the non-specific tissue in-growth into the polymer and then placed in one window and covered with Interceed while the other is not covered with filter before grafting. The scaffolds are then harvested after 1, 2, 3, 4, and 6 weeks. The molecular weight as well as morphology, thickness and weight of the scaffolds are evaluated as described above for in vitro experiment.

In vivo host reaction to implanted scaffolds—Inflammation is characterized by a local reaction that may be followed by the activation of an acute phase reaction. Some inflammatory markers can indicate the severity of inflammation, and their levels can be associated with the type of the polymer from which the scaffolds are constructed as well as the release of its degradation products. Rather than being a detrimental effect, this inflammatory response may be of some benefit because leukocytes that have migrated into the scaffold will release a plethora of growth factors that will lead to further tissue infiltration.

Detection of inflammatory cells—After scaffold harvesting, they are frozen-embedded with Tissue-tek in liquid nitrogen and sectioned using a cryostat. All cell nuclei are counterstained using haematoxylin-eosin. For detection of inflammatory cells, Giemsa staining is performed at 45° C. for 30 min and differentiated in 1% acetic acid solution. In Giemsa staining, the negatively charged phosphoric acid groups of DNA attract the purple polychromatic dyes. The blue basophilic granules are stained by the polychromatic cationic dyes. Cationic cellular components such as erythrocytes and eosinophilic granules are stained by red and pink anionic dyes.

Fibrinogen determination—Fibrinogen is considered not only as a coagulation component, but also an inflammatory marker. For its determination, the coagulative method of Clauss is used: high-sensitivy C-reactive protein (hs-CRP) is determined by the nephelometric method.

Statistical analysis—All data are arranged as mean ± standard deviation. Significant differences are determined using analysis of variance (ANOVA) and Fisher's least significant difference test as needed. Significance is reported at the 0.05 level.

Testing of Scaffold Biocompatibility: In Vitro Culture of Isolated Primordial and Primary Follicles

In order to test the biocompatibility of the scaffold(s) towards ovarian follicles, human follicles are seeded in the scaffolds and cultured in vitro for 7 days. In this first experiment, isolated follicles are used, while in the second experiment (see below), small cubes of ovarian tissue containing follicles are used.

Collection of the ovarian tissue—The use of human tissue for this study was approved by the Institutional Review Board of the Universite Catholique de Louvain. After obtaining written informed consent, an ovarian biopsy is taken from a woman between 20 and 30 years of age. The biopsy is divided into 2 fragments: one is used for follicle isolation and the other is cut in three pieces (control 1)—one piece is fixed in formalin for apoptosis, proliferation and follicular population studies, the second piece is fixed in Karnovsky fixative (2% paraformaldehyde and 2.5% glutaraldehyde in 0.1 M sodium cacodylate buffer—pH 7.4) to assess follicle morphology through transmission electron microscopy (TEM) and the last piece is frozen-embedded with Tissue-tek in liquid nitrogen for mitochondria activity assay.

Ovarian follicle isolation—The protocol previously described by Dolmans et al., (Hum. Reprod, 21:413-420, 2006) is used to isolate primordial and primary follicles. Briefly, the cortical portion of the ovary is placed in a tissue chopper, adjusted to 0.5 mm. The obtained ovarian fragments are transferred to 50 ml conical flasks containing 10 ml of PBS supplemented with 0.04 mg/ml Liberase blendzyme 3 and incubated in a water bath at 37° C. for 75 min with gentle agitation. The ovarian digest is periodically (every 15 min) agitated by a pipette to mechanically disrupt digested tissue. Digestion is terminated by the addition of an equal volume of PBS at 4° C. supplemented with 10% fetal bovine serum.

Ovarian follicle recovery—After enzyme inactivation, the suspension is centrifuged at 50×g for 10 min at 4° C. and the pellet containing the follicles is resuspended in 7.5 ml of Ficoll solution (density=1.1 g/cm³) at the bottom of a 50 ml conical flask, constituting the first Ficoll density layer. The successive density layers are subsequently added on top to complete the discontinuous gradient: 3.5 ml of 1.09 g/cm³ Ficoll solution, and 2.5 ml of 1.06 g /cm³ Ficoll solution and 2.5 ml of PBS. The gradient flask is centrifuged at 50×g for 17 min at 4° C. Finally, the interface between Ficoll 1.09 and Ficoll 1.06 as well as between Ficoll 1.06 and PBS is transferred to a Petri dish in order to recover the isolated follicles. The recovered isolated follicles (and also partially isolated follicles) are then divided into 3 aliquots: one for in vitro culture and the others (control 2) for metabolic activity assay and TEM analysis.

Embedding of isolated follicle in plasma clot for in vitro culture—Isolated primordial and primary follicles are then embedded in plasma clots (control 3) according to the following procedure: the patient's blood is centrifuged at 405 g for 15 min at 4° C. and the supernatant is recovered. Isolated follicles are injected in a droplet of 20 μl of this fresh plasma and the clot is induced by adding a droplet of 0.025 M CaCl₂, followed by incubation at 37° C. for 30 min.

In vitro culture of the isolated follicles—Follicles embedded in plasma clot as well as seeded in the scaffolds are then cultured using a procedure reported by Carlsson et al. (Hum. Reprod,21:2223-2227, 2006). Briefly, a clot or a scaffold is placed in one of the wells from a 24-well plates fitted with inserts of 0.4 μm pore size and covered with 500 μl of minimal essential medium supplemented with 10% human serum albumin, 0.5 IU/ml recombinant human FSH, 1.1 mg/ml 8-bromoguanosine 3′,5′-cyclic monophosphate, 1% insulin, transferring and selenium (ITS) and 0.5% antibiotic/antimycotic. Every second day, 110 μl of the culture medium is removed and replaced with fresh medium. The follicles are cultured for 7 days at 37° C. in a 5% CO₂ humidified environment and at the end of the culture period, the clots and scaffolds are destined to morphology analysis, metabolic and mitochondria activity assays, apoptosis or proliferation evaluation.

Morphology analysis—Micrographs are obtained in a SEM and TEM to study temporal, microscopic, structural changes in the scaffolds as well as the isolated follicles during in vitro culture. For SEM, the samples are dehydrated through a series of graded alcohols and then, are critical point dried. Finally, the samples are gold-sputtered at 20 mA for a total time of 120 seconds (coating thickness, approximately 40 nm). Then, they are imaged with a scanning electron microscope operated at 20 kV. For TEM, the specimens are rinsed in buffer and post-fixed in 1% osmium tetroxide, 0.8% potassium ferricyanide and 5 mm CaCl₂ in 0.1 M sodium cacodylate buffer for 1 h, followed by block staining in 0.5% uranyl acetate. Subsequently, the samples are dehydrated in acetone and then embedded in Spurr epoxy resin. Thin sections (70 nm) are contrasted with uranyl acetate and lead citrate, and examined using a transmission electron microscope.

Apoptosis assessment—Samples fixed in formalin are embedded in paraffin and 5 μm sections are cut from the blocks and air-dried on slides. Apoptosis is then analysed by a terminal deoxynucleotidyl transferase (TdT)-mediated biotinylated deoxyuridine triphosphates (dUTP) nick end-labelling (TUNEL) technology method to detect DNA fragmentation, and by immunohistochemistry for active caspase-3 to detect cells programmed to undergo apoptosis. For TUNEL, sections have been dewaxed with histosafe, rehydrated with isopropanol, and washed in running deionised water. The slides are then pretreated with 20 μg/ml of proteinase K working solution in 10 mM Tris-HCl (pH 7.5) for 30 min at 37° C. in a humidified chamber. Strand breaks of DNA occurring during the apoptotic process are detected by means of the In Situ Cell Death Detection Kit, TMR Red, a TUNEL assay. After washing with PBS, slides are incubated with a TUNEL reaction mixture: 50 μl enzyme solution (terminal deoxynucleotidyl transferase) and 450 μL label solution (nucleotide mixture in reaction buffer) for 60 min at 37° C. in a humidified chamber protected from light, followed by rinsing with PBS. Positive control sections are treated with 1,500 U/ml DNase I in 50 mM Tris-HCl (pH 7.5) 1 mg/mL bovine serum albumin (BSA), for 10 min at room temperature (RT) in a humidified chamber, before incubation with the TUNEL reaction mixture. Negative control sections are incubated with label solution without enzyme solution. Finally, slides are covered with Vectashield Mounting Medium with 4′,6-diamino-2-phenylindole (DAPI). This special formulation is intended to preserve fluorescence during prolonged storage and, at the same time, to counterstain DNA by means of DAPI. Slides are then coverslipped and sealed around the perimeter with nail polish, stored at 4° C., and protected from light until examination. TUNEL-stained and DAPI-counterstained slides can be examined under an inverted fluorescence microscope. Red fluorescence could be visualized in TUNEL-positive cells with the use of an excitation wavelength in the range of 520-560 nm, and by observing the emitted light at a wavelength between 570-620 nm. DAPI reached excitation at about 360 nm, and emitted at about 460 nm when bound to DNA, producing a blue fluorescence in all nuclei. Morphometric analysis of TUNEL-positive surface area is then performed to quantify apoptosis. For this purpose, sections are examined at X200 magnification, and all highpower fields (HPFs) are digitalized, either for TUNEL staining or DAPI counterstaining. ImageJ is used to delimit all TUNEL-positive cells and to measure their surface area, as well as to determine total surface area in each section (by measuring DAPI-counterstained surface area). The active caspase-3 technique is an immunohistochemical assay for the detection of the enzyme caspase-3, which can be activated during the apoptotic process and which, in turn, eventually activates endonucleases that cause the characteristic morphology of apoptotic cells. After deparaffination and rehydratation of slides as already described, an immunoperoxidase method is performed. Briefly, slides are treated with 0.3% H₂0₂ for 30 min at RT to inactivate endogenous peroxidase activity, heated in a solution of 10 mM sodium citrate at 95° C. for 75 min to retrieve epitopes, and incubated with 10% normal goat serum and 1% BSA in Tris-buffered solution for 30 minutes at RT to block non-specific staining. The slides are then incubated in a 1:100 dilution of the primary antibody, an anti-human rabbit polyclonal antibody directed against a peptide from the p18 fragment of human caspase-3 for 16 hours at RT. They are subsequently incubated with a secondary antibody conjugated to peroxidase, EnVision⁺® System Labelled Polymer-HRP Anti-Rabbit, for 2 hours at RT. The presence of peroxidase is then revealed by incubating with Liquid DAB+Substrate Chromogen System for 15 min at RT. Human menstrual endometrium can be used as a positive control. Slides are counterstained with haematoxylin.

Follicular proliferation assay—This assay is important to observe the recruitment and growth of the follicles during in vitro culture that is shown by the percentage of follicles with Ki-67-positive granulosa cells. Ki-67 is a nuclear antigen associated with cell proliferation and is present throughout the active cell cycle (late G1, S, G2, and M phases) but absent in resting cells (GO). Results are analysed according to the follicular stage in the three different groups. In order to facilitate identification of follicles, immunohistochemical analysis of inhibin-α are performed. Inhibin has two isoforms, a and β, with the same α-subunit but different β-subunits. Inhibin-α subunit is detected in granulosa cells at all follicular stages. Embedded sections are deparaffinized with Histosafe and rehydrated in 2-propanol. Endogenous peroxidase activity is blocked by incubating the sections with 0.3% H₂O₂ for 30 min at room temperature. The sections are decloaked in citrate buffer for 75 min at 98° C. before incubation with goat serum to block non-specific binding sites for 30 min and are then incubated overnight with primary antibodies: rabbit anti-human Ki-67 IgG, mouse monoclonal anti-human inhibin-α IgG (room temperature, 1:10 dilution). The slides are subsequently incubated for 60 min at RT with secondary antibodies: goat anti-rabbit or goat anti-mouse (1:2 dilution). Diaminobenzidine (Dako) is used as a chromogen and nuclei are counterstained with haematoxylin. Human proliferative endometrium is used as a positive control for Ki-67 labelling and human placental tissue for inhibin-α staining.

To assess the viability of the isolated follicles or the ovarian tissue in the scaffold, the following viability assays are used.

1. Fluorescent staining—Viability is analysed by vital fluorescent staining (calcein-AM and ethidium homodimer-1). Nonfluorescent cell-permeant calcein-AM enters the cell and is cleaved by non-specific esterase activity in living cells, producing calcein. The polyanionic dye, calcein, is well retained within live cells, giving an intense uniform green fluorescence, which can be visualized after exposing the tissue to light with a wavelength of 495 nm and observing the emitted light at a wavelength of 515 nm. Ethidium homodimer-I enters permeable cells (cells with damaged membranes) and then binds to

DNA with high affinity, undergoing a 40-fold enhancement of fluorescence, thereby producing bright red fluorescence in dead cells. In this assay, ovarian tissue, as well as scaffolds and plasma clots containing the isolated follicles, are cut into strips of 200 to 300 μm in thickness. Then, they are washed in Dulbecco PBS (DPBS) and exposed to 2 mM of calcein-AM in DPBS for 45 minutes at 37° C. in the dark. Five mM of ethidium homodimer-1 is added to counterstain the nuclei of all dead cells. After exposure, the tissue strips are washed in DPBS, mounted between coverslips, and evaluated under an inverted fluorescence microscope. The cytoplasm of all live cells appears bright green. Follicles show up as bright green large dots in the more weakly stained interstitial tissue.

2. Metabolic activity assays—This is another assay to assess follicular viability after in vitro culture. Ovarian follicles are rinsed with ice-cold homogenisation buffer (10 mM Tris.HCl, pH 7.0, 0.25 M sucrose, 10% glycerol) supplemented with 1 mM PMSF and 10 μg/ml each of pepstatin, antipain, soybean trypsin inhibitor and benzidine-HCl to minimise proteolysis. They are then homogenised in 35 μl homogenisation buffer and the homogenate is centrifuged at 26000 g for 30 min in an eppendorf microfuge at 4° C. to separate mitochondrial fraction. The supernatant is used to determine the activities of phosphofructokinase (PFK) and pyruvate kinase (PK), two key regulatory enzymes of glycolysis. The pellet is resuspended in homogenisation buffer to determine the activity of malate dehydrogenase (MDH), an important enzyme of the Krebs cycle.

Subsequently, the PFK activity is determined as follows: The reaction mixture containing 33 mM Tris.HCl, pH 8.0, 2 mM ATP, 5 mM MgSO₄2 mM fructose-6-phosphate (potassium salt), 0.16 mM NADH, 1 mM dithiothreitol, 0.05 mM KCl and 66.6 μl of an auxiliary enzyme solution (aldolase, triose phosphate isomerase and glycero-phosphate dehydrogenase) is incubated at 37° C. in a temperature-controlled quartz cuvette and absorbance is recorded in a spectrophotometer. After recording the background rate of NADH oxidation for 5 min without samples, 10 μl of supernatant is added to the reaction mixture, mixed and the rate of NADH oxidation is recorded at 1-min intervals of 5 min. The enzyme activity can then be expressed in millimoles NADH oxidised per minute per milligram protein.

Next, the PK activity is analysed as follows: The reaction mixture containing 50 mM triethanolamine buffer, pH 7.5, 2.5 M KCl, 0.24 M MgSO₄, 6 μM ADP, 18 U/ml lactic dehydrogenase, 1.4 μmol NADH and 5 μl of follicular supernatant is recorded at 340 nm at 37° C. After recording of the background rate of NADH oxidation for 5 min without substrate, 45 mM phosphoenol pyruvate is added to the mixture and mixed immediately. The rate of NADH oxidation is then recorded at 1-min intervals for 5 min. The enzyme activity can be expressed as millimoles NADH oxidized per minute per milligram protein.

Finally, the MDH enzyme activity is determined from the following reaction mixture containing 100mM potassium phosphate buffer, pH 7.5, 50 mM oxaloacetate and 20 mM NADH. The rate of NADH oxidation following the addition of follicular pellet fraction is recorded as described above and the activity is expressed in millimoles NADH oxidized per minute per milligram protein.

3. Mitochondrial hydroxylase enzymatic activity test (MU test)—This assay also helps to assess follicular viability and is performed as described by Obal et al. (Anesth Analg, 101:1252-1260, 2005). Briefly, the frozen samples are cut into 8 pm sections and the slides are incubated for 15 min in buffered 1% triphenyltetrazoliumchloride (TTC) (pH 7.4) at 37° C. and then fixed in formaldehyde for 48 h. Viable follicles are identified as red stained by TTC, whereas dead follicles appear pale grey.

4. Anti-Müllerian hormone (AMH) measurement—It has been suggested that AMH might act as a survival factor for the small growing follicles, preventing them from undergoing atresia. Therefore, its level in the culture medium can be correlated to the follicle survival. To measure AMH concentration, the culture medium that was removed every second day during in vitro culture is stored at −80° C. until assayed by second-generation ELISA, according to the protocol described by Fanchin et al. (J Clin Enddocrinol Metab, 92:1796-1802, 2007). Levels of AMH are expressed as nanograms per gram of protein.

Testing of Scaffold Biocompatibility: In Vitro Culture of Small Cubes of Ovarian Tissue Containing Primordial and Primary Follicles

Collection of the ovarian tissue—The use of human tissue for this study was approved by the Institutional Review Board of the Universite Catholique de Louvain. After obtaining written informed consent, ovarian biopsies were taken from women between 20 and 30 years of age. The biopsies are divided into 2 fragments: one used for in vitro culture and the other to be cut in three pieces (control 1)—one piece fixed in formalin for apoptosis, proliferation and follicular density studies, another was fixed in Karnovsky fixative to assess follicle morphology through TEM and the last one was frozen-embedded with Tissue-tek in liquid nitrogen for mitochondria activity assay.

In vitro culture of ovarian tissue—In vitro culture is performed according to the procedure reported by Carlsson et al. (Hum. Reprod,21:2223-2227, 2006): the ovary fragment is cut in small cubes (approximately 1-2 mm³) and divided into two groups. One group is seeded in the scaffold and the other not. Then, they are placed in a 24-well plate (2-5 cubes/well) fitted with 0.4 μm inserts and covered with 500 μl of minimal essential medium supplemented with 10% human serum albumin, 0.5 IU/ml recombinant human FSH, 1.1 mg/ml 8-bromoguanosine 3′,5′-cyclic monophosphate, 1% ITS and 0.5% antibiotic/antimycotic. Every second day, 110 μl of the culture medium is removed and replaced with fresh medium.

The ovarian tissue is then cultured for 7 days at 37° C. in a 5% CO₂ humidified environment and at the end of the culture period, destined to morphology analysis, metabolic and mitochondria activity assays, apoptosis or proliferation evaluation as previously described for cultured isolated follicles.

Statistical analysis—The proportions of follicles at different developmental stages, density of follicles and proportions of viable follicles are then analysed. Significant differences are determined using analysis of variance (ANOVA) and Fisher's least significant difference test as needed. Significance is reported at the 0.05 level.

Testing Scaffold Bioactivity: Short-Term Grafting of Ovine Primordial and Primary Follicles

It is known that many biologically functional molecules, extracellular matrix components, and cells interact at the nanoscale and this creates a highly specialized microenvironment, which is essential for correct cell development and continued function. For this reason, in order to induce and coordinate folliculogenesis in the patient graft, it is necessary to program the scaffold with delivery of bioactive molecules, such as factors that may positively influence neovascularisation, follicle growth and development and oocyte maturation. These factors are encapsulated in nanospheres to protect them from denaturation that could occur if they are directly adsorbed onto the scaffold, which would result in complete degradation of them during a very short release time. The released amount of factors can be modulated by the encapsulated amount of factors in the nanospheres, the amount of nanospheres incorporated in the scaffold or the composition of the nanospheres. Therefore, nanospheres containing different factors implied in folliculogenesis as well as factors that may reduce ischaemic damages and angiogenesis factors can be tested. Nanospheres are built using the same techniques and polymers (and its copolymers) previously described above and loaded with different factors:
Factors involved in the primordial follicle or preantral follicle development:
   Activin;
   Basic fibroblast growth factor (bFGF);
   Kit ligand;
   Insulin;
   Bone morphogenetic protein—4 (BMP-4);
   Bone morphogenetic protein—7 (BMP-7);
   Leukaemia inhibitory factor (LIF) ;
   Nerve growth factor (NGF);
   Keratinocyte growth factor (KGF);
   Growth differentiation factor—9 (GDF-9) necessary in primary follicle development and it is present in primary to antral follicles;
   17α hydroxylase (17α-OH) involved in the differentiation of fibroblastic cells around the follicle to theca cells.
   The most preferred candidate factors in this group are GDF-9 and/or 17α-OH.
Anti-Müllerian Hormone (AMH) and/or stromal cell-derived factor 1 (SDF-1): are both negative regulators of early follicle development; inhibiting primordial follicle recruitment.
Factors that reduce ischaemic damages:
   Ascorbic acid;
   Vitamin E;
   Pentoxifylline.
Factors involved in angiogenesis:
   Vascular endothelial growth factor (VEGF): it is a potent and specific stimulator of vascular endothelial cell proliferation and it also has permeability actions and may act as survival factor for immature vessels;
   Platelet-derived growth factor: it also regulates angiogenesis;
   Angiopoietins: it enhances the maturation and stabilization of newly formed blood vessels. Angiopoietin-1 which specifically binds to and stimulates the TIE-2 receptor is a marker of active neovascularisation process.
   Placenta growth factor (PIGF): it stimulates angiogenesis, including growth of collateral vessels in non-healthy tissue.
   HIF polyl hydroxylases (PHD1): they are oxygen sensors that regulate the stability of HIFs. They provide protection against lethal ischemia.
   Hypoxia mimic ions: they promote angiogenesis, establish a functional vasculature and activate cell differentiation, cytoprotective properties, lymphangiogenesis and progenitor cell recruitment.
   PR39: it is a macrophage derived peptide, inhibited the ubiquitin-proteosome-dependent degradation of hypoxia-inducible factor is protein (HIF-1α), resulting in accelerated formation of vascular structures in vitro.
   P53: it directly interacts with HIF-1α and limits the hypoxia-induced expression of HIF-1αa by stimulating Mdm2-mediated ubiquination and proteasomal degradation under hypoxic conditions.
   Interleukin-8 (IL-8): it is a chemoattractant and activating factor for human neutrophils and a potent angiogenic agent. It is one of the most important cytokine in ovarian angiogenesis.
   Transforming growth factor-β1 (TGF-β1): it is known to be important in regulating angiogenesis. In the ovary, it has been showed that TGF-β1 levels increase during revascularization following transplantation, which supports a role for this factor in regulating vascular function.
   Nitric oxide (NO): it is known to mediate physiological functions, such as vasodilation, regulation of angiogenesis, and blood flow in many tissue, including the ovary. The presence of exogenous NO supports HIF-1α stabilization.

In a preferred embodiment, the combination of factors comprises one factor of each of the following groups:
Factors involved in the primordial follicle or preantral follicle development, the most preferred candidate factors in this group being Growth differentiation factor-9 (GDF-9) and/or 17α-OH;
Anti-Müllerian Hormone (AMH) and/or stromal cell-derived factor 1 (SDF-1);
Factors that reduce ischaemic damages; and
Factors involved in angiogenesis.

In a more preferred embodiment, the combination of factors is as follows:
Factor involved in the primordial follicle or preantral follicle development: Growth differentiation factor-9 (GDF-9)
Inhibitor factor: Anti-Müllerian Hormone (AMH)
Factor that reduce ischaemic damages: Ascorbic acid
Factors involved in angiogenesis: HIF polyl hydroxylases (PHD1): they are oxygen sensors that regulate the stability of HIFs. They provide protection against lethal ischemia.

In order to test the influence of these factors, scaffolds containing primordial and primary follicles have been grafted in adult ewes for three weeks. One experiment is performed to implant scaffolds containing isolated follicles or small cubes of ovarian tissue. An evaluation of the host reaction to the scaffold is performed with the aim to investigate if the degree of inflammation is related with the level of vascularisation of implants through angiogenesis.

Collection of the ovarian tissue—Ovaries from adult ewes are used in this experiment. For this, a laparotomy is performed to remove the right ovary. In the laboratory, the ovary is divided into 3 fragments: the first fragment is used for follicle isolation, the second is cut into small cubes and the third is cut in three pieces (control 1)—one piece is fixed in formalin for apoptosis, proliferation, follicle density and vascularisation studies, other is fixed in Karnovsky fixative to assess follicle morphology through TEM and the last one is frozen-embedded with Tissue-tek in liquid nitrogen for mitochondria activity assay.

Ovarian follicle isolation and recovery—primordial and primary follicles are isolated and recovered as previously described in the second part of this study. The recovered isolated follicles (and also partially isolated follicles) are divided into 3 aliquots: one for grafting in the scaffold and the others (control 2) for metabolic activity assay and TEM analysis.

Scaffold grafting—For the grafting of isolated follicles seeded in the scaffold as well as the small cubes of ovarian tissue enclosed in the scaffold, a laparotomy is performed as described previously in this study. After three weeks, another laparotomy is performed in order to remove the sheep ovary containing the scaffolds. Analysis of the scaffold/follicle morphology as well as assessment of follicle viability, apoptosis, metabolic and mitochondrial activity, cell proliferation and host reaction to the scaffold are carried out as previously described.

The following tests are then used to establish whether the scaffold comprising the isolated follicles or the small cubes of ovarian tissue is capable of inducing angiogenesis needed for the survival and development of the primary follicles into mature follicles:

1. Vascular Endothelial Growth Factor—Immunohistochemical assays are performed on formalin-fixed, paraffin-embedded 5 μm sections. The sections are deparaffinized in histosafe and rehydrated through graded isopropanol. Then, they are incubated with 0,3% H₂O₂ for 30 min at RT to eliminate endogenous peroxidase. The slides are incubated for 20 min at 96° C. in TRIS 10 mM +EDTA 1 mM pH 9.0 for antigen retrieval, rinsed in TBS and blocked with TBS, 10% NGS, 1% BSA for 30 min at RT. After that, they are incubated overnight at 4° C. with Mouse anti-HuVEGF diluted 1:50 in TBS, 1% NGS, 0.1% BSA and rinsed in TBS. Primary antibodies are developed with DAKO EnVision anti-mouse kit coupled with streptavidin-horseradish peroxidase (HRP) following the manufacturer instruction, stained using 3,3′-diaminobenzidine (DAB), and counterstained with haematoxylin.

2. CD34—Immunohistochemical assays are performed on formalin-fixed, paraffin-embedded 5-μm sections. The sections are deparaffinized in histosafe and rehydrated through graded isopropanol. Then, they are incubated with 0,3% H₂O₂ for 30 min at RT to eliminate endogenous peroxidase. The slides are rinsed in TBS and blocked with TBS, 10% NGS, 1% BSA for 30 min at RT. Then, they are incubated overnight at 4° C. with mice anti anti-human CD34 diluted 1:8000 in TBS, 1% NGS, 0,1% BSA and rinsed in TBS. Primary antibodies are developed with DAKO EnVision anti-mouse kit coupled with streptavidin-horseradish peroxidase (HRP) following the manufacturer instruction, stained using 3,3′-diaminobenzidine (DAB), and counterstained with haematoxylin.

3. Angiopoietin-1 (Ang-1)—Immunohistochemical detection of Ang-1 is carried out on sections from paraffin-embedded tissues using streptavidin-biotinylated HRP detection. Antigen retrieval is performed by heating of tissue sections in a microwave oven for 10 min, and non-specific binding is prevented by incubation with PBS containing 2% BSA (PBSA). Tissue sections are incubated with Tie-2/Fc chimera diluted to 5 μg/ml in 2% PBSA containing 0.6% Triton X. Human IgG1 Fc is then used as a control for Tie-2/Fc. 3,3′-Diaminobenzidine is used as a chromogen, and sections are subsequently counterstained with haematoxylin or toluidine blue.

4. α-Smooth muscle actin (αSMA)—For detection of pericytes and vascular smooth muscle cells, sections are stained with monoclonal αSMA antibody, conjugated to alkaline phosphatase and visualised with Fast Red. The slides are counterstained with Mayer's haematoxylin solution.

Statistical analysis—The effect of the presence of different factors on the percentage of normal follicles is then analysed by ANOVA. Fisher's PLSD post hoc test is then used to make individual comparisons between each treatment and the controls and among treatments. Percentages are transformed to arcsine √ % prior to analysis. The percentages of normal follicles on Day 0 (control) and Day 21 (last day of grafting) are compared among treatments by chi-square test with Yate's correction. Data are presented as mean ±standard deviation and significance is reported at the 0.05 level.

Testing Scaffold Bioactivity: Scaffold Long-Term Grafting

After all the previous studies to determine the best scaffold to graft isolated follicles as well as small cubes of ovarian tissue, it is also important to test the long-term grafting of the scaffold to observe its degradability and its ability to assist follicular growth in the host. Another important issue to address is the capacity of frozen follicles to survive and develop in such scaffolds. In order to answer these questions, a last part of this study are carried out. As for some of the previous parts, two experiments are carried out: one for isolated follicles and other for small cubes of ovarian tissue. For long-term grafting of isolated primordial and primary follicles, the following steps are performed:

Collection of the ovarian tissue—After obtaining written informed consent, ovarian biopsies are taken from women between 20 and 30 years of age. The biopsies are divided into 2 fragments: one is used cut into two pieces—one piece is used for follicle isolation and the other piece is frozen as described below. The other fragment is cut in three pieces (control 1)—one piece is fixed in formalin for apoptosis, proliferation, follicle density and vascularisation studies, other is fixed in Karnovsky fixative to assess follicle morphology through TEM and the last one is frozen-embedded with Tissue-tek in liquid nitrogen for mitochondria activity assay.

Ovarian tissue freezing and thawing—Freezing of the ovarian tissue fragments is performed according to the method described by Gosden et al. (Hum Reprod, 9:597-603, 1994) with some modifications. The tissue is first suspended in 800 μl of MEM-Hepes in a cryovial. Then, this medium is replaced with the same amount of the cryopreservation solution (10% DMSO and 2% HSA in MEM-Hepes) at 0° C. The cryovials are cooled in a programmable freezer with the following program: (1) cooled from 0° C. to −8° C. at −2° C./min; (2) seeded manually by touching the cryovials with forceps prechilled in liquid nitrogen; (3) cooled to −40° C. at −0.3° C./min, and transferred to liquid nitrogen (−196° C.) for storage. The cryovials are thawed at RT for 2 min and immersed in water at 37° C. until the ice is completely melted. To remove the cryoprotectant solution, the ovarian tissue is transferred from the cryovials to Petri dishes containing MEM-Hepes, where it is washed three times (5 min each bath) before follicle isolation or grafting (second experiment).

Ovarian follicle isolation and recovery—primordial and primary follicles are isolated and recovered as previously described in the second part of this study. The recovered isolated follicles (and also partially isolated follicles) are divided into 3 aliquots: one for grafting in the scaffold and the others (control 2) for metabolic activity assay and TEM analysis. Grafting of the scaffolds is performed as previously described. After 24 weeks, the grafts are removed and the same analysis described in the first experiment of the third part of this study are carried out.

Sheep immunosuppression and scaffold grafting—Cyclosporine is used for immunosuppression of the animals, according to the method described by Rose et al. (Immunol Immunopath, 81:23-36, 2001). For the grafting, a laparotomy is performed as described previously in this study. After 24 weeks, another laparotomy is performed in order to remove the sheep ovary containing the scaffold. Analysis of the scaffold/follicle morphology as well as assessment of follicle viability, apoptosis, metabolic and mitochondrial activity, cell proliferation and host reaction to the scaffold is carried out as previously described.

Similarly, long-term grafting of small cubes of ovarian tissue containing primordial and primary follicles is also tested. After obtaining written informed consent, ovarian biopsies are taken from women between 20 and 30 years of age. The biopsies are divided into 2 fragments: one is used cut into two pieces—one piece is used for grafting into the scaffold and the other piece is frozen as described below. The other fragment is cut in three pieces (control 1)—one piece is fixed in formalin for apoptosis, proliferation, follicle density and vascularisation studies, other is fixed in Karnovsky fixative to assess follicle morphology through TEM and the last one is frozen-embedded with Tissue-tek in liquid nitrogen for mitochondria activity assay. The grafting and analysis described before for isolated follicles is also performed for the grafting of ovarian tissue.

### EXAMPLES

The invention is illustrated by the following non-limiting examples.

### Example 1

### Isolation of ovarian primordial follicles from a patient.

Collection of the ovarian tissue—After obtaining written informed consent, ovarian biopsies are taken from women between 20 and 30 years of age. The biopsies are divided into 2 fragments: one is used cut into two pieces—one piece is used for follicle isolation and the other piece is frozen as described below. The other fragment is cut in three pieces (control 1)—one piece is fixed in formalin for apoptosis, proliferation, follicle density and vascularisation studies, other is fixed in Karnovsky fixative to assess follicle morphology through TEM and the last one is frozen-embedded with Tissue-tek in liquid nitrogen for mitochondria activity assay.

Ovarian tissue freezing and thawing—For the freezing of the ovarian tissue fragments, the tissue is first suspended in 800 μl of MEM-Hepes in a cryovial. Then, this medium is replaced with the same amount of the cryopreservation solution (10% DMSO and 2% HSA in MEM-Hepes) at 0° C. The cryovials are cooled in a programmable freezer with the following program: (1) cooled from 0° C. to −8° C. at −2° C./min; (2) seeded manually by touching the cryovials with forceps prechilled in liquid nitrogen; (3) cooled to −40° C. at −0.3° C./min, and transferred to liquid nitrogen (−196° C.) for storage. The cryovials are thawed at RT for 2 min and immersed in water at 37° C. until the ice is completely melted. To remove the cryoprotectant solution, the ovarian tissue is transferred from the cryovials to Petri dishes containing MEM-Hepes, where it is washed three times (5 min each bath) before follicle isolation or grafting (second experiment).

Ovarian follicle isolation and recovery—primordial and primary follicles are isolated and recovered as previously described. The recovered isolated follicles (and also partially isolated follicles) are divided into 3 aliquots: one for grafting in the scaffold and the others (control 2) for metabolic activity assay and TEM analysis. Grafting of the scaffolds is performed as previously described. After 24 weeks, the grafts are removed and the follicles are analysed for their viability and developmental state as described above.

### Example 2

In vitro culturing of isolated ovarian primordial follicles or ovarian tissue and analysis of viability and developmental status of follicle cells.

Embedding of isolated follicle in plasma clot for in vitro culture—Isolated primordial and primary follicles are then embedded in plasma clots following the method described by

Gosden et al (Hum Reprod, 5:499-504, 1990). In short, the patient's blood is centrifuged at 405 g for 15 min at 4° C. and the supernatant is recovered. Isolated follicles are injected in a droplet of 20 pl of this fresh plasma and the clot is induced by adding a droplet of 0.025 M CaCl₂, followed by incubation at 37° C. for 30 min.

In vitro culture of the isolated follicles—Follicles embedded in plasma clot as well as seeded in the scaffolds are then cultured using a procedure reported by Carlsson et al. (Hum. Reprod,21:2223-2227, 2006): a clot or a scaffold is placed in one of the wells from a 24-well plates fitted with inserts of 0.4 μm pore size and covered with 500 μl of minimal essential medium supplemented with 10% human serum albumin, 0.5 IU/ml recombinant human FSH, 1.1 mg/ml 8-bromoguanosine 3′,5′-cyclic monophosphate, 1% ITS (with a final concentration of 10 μg insulin/ml; 5.5 μg transferring/ml; 6.7 ng sodium selenite/ml) and 0.5% antibiotic/antimycotic. Every second day, 110 μl of the culture medium is removed and replaced with fresh medium. The follicles are cultured for 7 days at 37° C. in a 5% CO₂ humidified environment and at the end of the culture period, the clots and scaffolds are destined to morphology analysis, metabolic and mitochondria activity assays, apoptosis or proliferation evaluation.

### Example 3

Seeding of ovarian primordial follicles or ovarian tissue in the scaffold and scaffold grafting and testing the biocompatibility of the scaffold with the isolated follicles

Scaffold seeding—For the seeding of the follicles or ovarian tissue, isolated follicles or small cubes of ovarian tissue are placed into the scaffold of the device of the invention and allowed to adhere to the temporary surrogate for the native extracellular matrix, which helps forming an ovarian-like structure, favouring cell migration, attachment, multiplication and vascularisation. In addition, the scaffold permits transport of oxygen, nutrients and degradation products. Prior to the implantation of the device into the patient, the device is cultured in vitro for a period long enough for allowing the isolated follicles to enter the meshwork of scaffolds, attach to it and remain viable, using appropriate culturing conditions as for the plasma clot described above.

Scaffold grafting—For the grafting of the scaffold comprising either isolated follicles seeded in the scaffold or small cubes of ovarian tissue enclosed in the scaffold, a laparotomy is performed as described previously in this study. After three weeks, another laparotomy is performed in order to remove the sheep ovary containing the scaffolds. Analysis of the scaffold/follicle morphology as well as assessment of follicle viability, apoptosis, metabolic and mitochondrial activity, cell proliferation and host reaction to the scaffold are carried out as previously described.

Sheep immunosuppression and scaffold grafting—Cyclosporine is used for immunosuppression of the animals. For the grafting, a laparotomy is performed as described previously in this study. After 24 weeks, another laparotomy is performed in order to remove the sheep ovary containing the scaffold. Analysis of the scaffold/follicle morphology as well as assessment of follicle viability, apoptosis, metabolic and mitochondrial activity, cell proliferation and host reaction to the scaffold are carried out as previously described.

The same procedure can be followed using biopsies of small cubes of ovarian tissue. The biopsies are divided into 2 fragments: one is used cut into two pieces—one piece is used for grafting into the scaffold and the other piece is first frozen as previously described and then grafted. The other fragment is cut in three pieces (control 1)—one piece is fixed in formalin for apoptosis, proliferation, follicle density and vascularisation studies, other is fixed in Karnovsky fixative to assess follicle morphology through TEM and the last one is frozen-embedded with Tissue-tek in liquid nitrogen for mitochondria activity assay. The grafting and analysis described before for isolated follicles are also performed for the grafting of ovarian tissue.

Due to their unique characteristics, the scaffolds of the invention can be implanted in a subject in need thereof. Due to the presence of the bio-activating and bio-inhibiting factors, the scaffold not only maintains viability of the follicles present in the scaffold, but induces and stimulates their development, amongst other by inducing neo-vascularisation inside the scaffold, enabling the transport of the plethora of (many yet unknown) factors and stimulants needed for efficient follicle development and maturation. After neo-vascularisation, all naturally present and yet largely unknown factors and signals are transported right to the follicles inside the scaffold, which cannot be mimicked in any in vitro model system provided in the prior art.

1. A device, comprising a scaffold composition consisting essentially of a flexible implantable biocompatible matrix with a porous structure, a bio-activating composition and a bio-inhibiting composition, wherein said bio-activating and bio-inhibiting composition are incorporated into or coated onto said scaffold composition, wherein said scaffold composition is biocompatible and biodegradable and temporally controls growth of resident primordial follicles, migration and multiplication of stroma cells and spreading and organization of endothelial cells and new vessels, wherein said bio-activating composition regulates positive development of said resident primordial follicles into primary follicles, formation of new blood vessels and chemoattraction and proliferation of stroma cells and wherein the bio-inhibiting composition inhibits the development of other resident primordial follicles into primary follicles.

2. The device according to claim 1, wherein said bio-activating composition and said bio-inhibiting composition are extracellular matrix components.

3. The device according to claim 1, wherein the bio-activating composition and/or the bio-inhibiting composition are encapsulated within a slow release container.

4. The device according to claim 1, wherein the bio-inhibiting composition comprises anti-Müllerian hormone (AMH) and/or stromal cell-derived factor 1 (SDF-1).

5. The device according to claim 1, wherein the bio-activating composition comprises growth differentiation factor-9 (GDF-9).

6. The device according to claim 1, wherein the bio-activating composition comprises one or more of activin, basic fibroblast growth factor (bFGF), Kit ligand, insulin, bone morphogenetic protein-4 (BMP-4), bone morphogenetic protein—7 (BMP-7), leukaemia inhibitory factor (LIF), nerve growth factor (NGF) and keratinocyte growth factor (KGF), 17α hydroxylase (17α-OH).

7. The device according to claim 1, wherein the bio-activating composition comprises one or more of factors reducing ischaemic damages such as ascorbic acid, vitamin E or Pentoxifylline.

8. The device according to claim 1, wherein the bio-activating composition comprises one or more of factors involved in angiogenesis such as vascular endothelial growth factor (VEGF), platelet-derived growth factor, angiopoietins such as Angiopoietin-1, placenta growth factor (PIGF), HIF polyl hydroxylases (PHD1) and hypoxia mimic ions, PR39, p53, interleukin-8 (IL-8), transforming growth factor-β1 (TGF-β1) and nitric oxide (NO).

9. The device according to claim 1, wherein at least one member of each of the following groups of factors is present:
a) factors involved in the primordial follicle or preantral development such as: activin, Basic fibroblast growth factor (bFGF), Kit ligand, Insulin, Bone morphogenetic protein—4 (BMP-4), Bone morphogenetic protein—7 (BMP-7), Leukaemia inhibitory factor (LIF), Nerve growth factor (NGF), Keratinocyte growth factor (KGF), Growth Differentiation Factor-9 (GDF-9) or 17α hydroxylase (17α-OH);
b) negative regulators of early follicle development: Anti-Müllerian Hormone (AMH) and/or stromal cell-derived factor 1 (SDF-1);
c) optionally, factors that reduce ischaemic damages such as Ascorbic acid, Vitamin E, or Pentoxifylline;
d) factors involved in angiogenesis such as: Vascular endothelial growth factor (VEGF), Platelet-derived growth factor, Angiopoietins, Angiopoietin-1, Placenta growth factor (PIGF), HIF polyl hydroxylases (PHD1), Hypoxia mimic ions, PR39, p53, Interleukin-8 (IL-8), Transforming Growth Factor-β1 (TGF-β1) and Nitric Oxide (NO).

10. The device according to claim 9, wherein the following factors are present in combination: one or more factors involved in the primordial follicle development selected from GDF-9 and/or 17α-OH; one or more negative regulators of early follicle development selected from Anti-Müllerian Hormone (AMH) and/or stromal cell-derived factor 1 (SDF-1); one or more factors that reduce ischaemic damages; and one or more factors involved in angiogenesis.

11. The device according to claim 10, wherein the following factors are present in combination: Growth differentiation factor—9 (GDF-9), Anti-Müllerian Hormone (AMH), Ascorbic acid and HIF polyl hydroxylases (PHD 1).

12. The device according to claim 1, wherein said scaffold composition comprises pores having a pore size between 10 and 6000 μm and/or wherein the pores are distributed within the scaffold in a controlled pattern, whereby the pores in the region of the centre of the scaffold are wider than the pores in the region towards the outer surface of the scaffold.

13. The device according to claim 1, wherein the device is provided with an inlet for the introduction of the follicles in the scaffold and/or, whereby the flexible implantable biocompatible matrix has a sufficient elasticity to allow follicle growth within the scaffold allowing the pores to adjust during growth from 10 to 6000 μm and/or wherein said device is cylindrical or suitable for use in a rolling-culture process in vitro.

14. The device of claim 1, wherein said device further comprises follicles.

15. The device of claim 1, which is constructed out of biodegradable material selected from the group consisting of: linear aliphatic polyesters: poly(lactic acid)—PLA, poly(glycolic acid)—PGA, poly(caprolactone)—PCL, poly(hydroxy butyrate)—PHB, including homopolymers and copolymers thereof, polyanhydrides, Poly(propylene fumarates) (PPF), Tyrosine-derived polymers, poly(ortho esters), poly(anhydrides), polyphosphazenes, polyurethanes, hydrogel matrices, alginic acid, hyaluronic acid, poly(γ-glutamic acid), amphiphiles, or combinations thereof.

16. A method of restoring fertility in a subject, comprising the implantation of a device according to claim 1.
